(19) United States
(12) Patent Application Publication
(10) Pub. No.: US 2006/0255291 A1
(43) Pub. Date: Nov. 16, 2006

(54) Sponge sterilizer

(76) Inventor: Debra J. Fogel Harris, Boca Raton, FL (US)

Correspondence Address: Debra J. Harris, 9366 Aegean Drive, Boca Raton, FL, 33496, US

(21) Appl. No.: 11/129,913

(22) Filed: May 16, 2005

(51) Int. Cl. A61L 2/10

(52) U.S. Cl. 250/455.11

(57) ABSTRACT

Disclosed is an apparatus for sterilizing sponges having a container, comprising a casing and lid, having a UV-transmissive support structure disposed therein, the UV-transmissive support dividing the interior of the container into a sponge chamber and a lamp chamber, the sponge chamber dimensioned to receive a sponge, a source of ultraviolet light disposed within the lamp chamber, and wherein at least one interior surface of the sponge chamber is reflective to UV light.

### BACKGROUND OF THE DISCLOSURE

1. Field of the Disclosure

This disclosure relates to an ultraviolet sterilizer for sponges.

2. Description of the Related Art

It is well known in the art of bacterial sterilization to use ultraviolet (UV) light to sterilize objects. Beasley et al., U.S. Pat. No. 4,906,851 for a U.V. Toothbrush Sterilizer and Toothbrush Holder, Issued Mar. 6, 1990, discloses a device for sterilizing and storing toothbrushes that includes a container for holding the toothbrushes whereby current to an ultraviolet ray lamp that supplies an application of germicidal radiation to bristles of toothbrushes stored therein can be automatically deenergized when the container is in an opened position at a predetermined distance.

Ettlinger et al., U.S. Pat. No. 6,811,748 for a System For and Method of Sterilization of Objects, issued Nov. 2, 2004, discloses a plurality of sources of ultraviolet light emission arranged in a disclosure formed so as to enclose an object to be sterilized with the sources of ultraviolet light emission emitting light into an inner chamber of the enclosure onto an object to be sterilized.

A drawback of the prior art is that the units are either not suited to the sterilization of household sponges or are simply too costly to manufacture for household use. What is needed is a relatively inexpensively manufactured device for sterilizing household sponges.

### BRIEF SUMMARY OF THE DISCLOSURE

Disclosed is an apparatus for sterilizing sponges having a container, comprising a casing and lid having a UV-transmissive support structure disposed therein, the UV-transmissive support dividing the interior of the container into a sponge chamber and a lamp chamber, the sponge chamber dimensioned to receive a sponge, a source of ultraviolet light disposed within the lamp chamber and wherein at least one interior surface of the sponge chamber is reflective to UV light.

In an embodiment of the invention, a glass window is provided to allow the user to see that the device is operational while blocking the user's exposure to short wave UV radiation.

### BRIEF DESCRIPTION OF THE DRAWINGS

FIG. 1 is a front cross-sectional view of the invention.

FIG. 2 is a side cross-sectional view of a first embodiment of the invention.

FIG. 3 is a side cross-sectional view of a second embodiment of the invention.

FIG. 4 is a front cross-sectional view of another embodiment of the invention.

### DETAILED DESCRIPTION OF THE PREFERRED EMBODIMENTS

Disclosed is an apparatus for sterilizing household sponges, which typically are highly germ-laden due to their repeated exposure to moisture and dirt. Household sponges typically remain moist after use, providing an excellent breeding ground for bacteria.

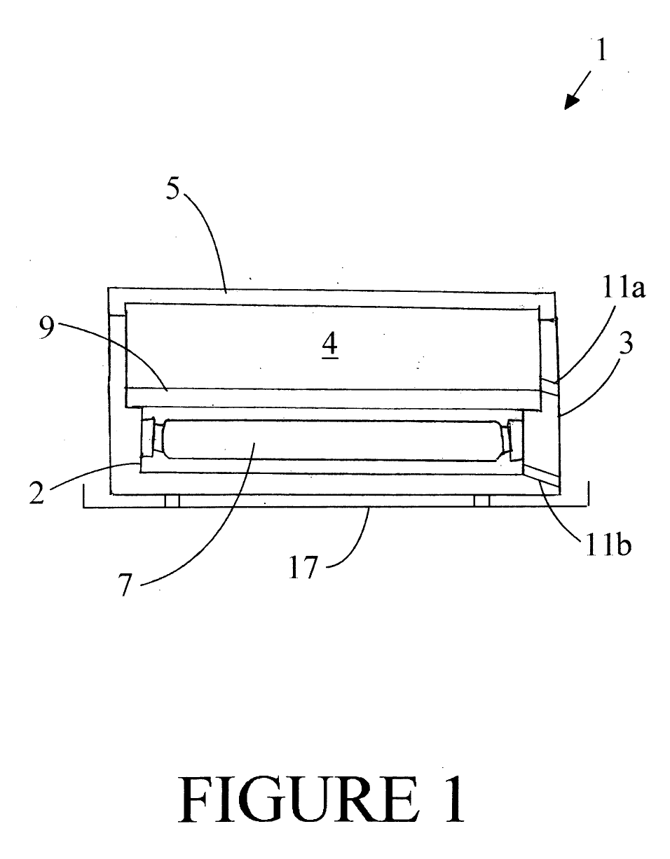

Referring to FIG. 1, the is shown a cross-sectional view of an embodiment of the sponge sterilizer 1 of the invention. A sponge chamber 4 and a lamp chamber 2 are defined by a casing 3 and lid 5, which are sized and dimensioned to hold short-wave UV lamp 7 in the lamp chamber and a sponge (not shown) in the sponge chamber 4. The two chambers are separated by a UV-transmissive support 9 for supporting the sponge over the UV lamp 7. The sponge chamber is dimensioned for typical household sponges and may be about 1 to 2 inches deep, 4 to 7 inches long, and about 4 to 6 inches wide, though of course these dimensions may be varied as desired.

The UV-transmissive support 9 may be a solid sheet of a UV-transmissive material, such as quartz, or a simple grid-like structure. If the UV transmissive support is chosen to be solid, then an upper drain hole 11a may be provided to drain away any fluids exuded from the sponge. If the UV-transmissive support is a grid-like structure, then one or more lower drain holes 11b may be provided for the same purpose. Generally, a grid-like structure may be preferred to reduce costs of manufacture. The drain holes 11 will preferable slope downward so as to substantially eliminate the possibility of exposure of a user to the UV radiation emitted by the UV lamp. A small collection tray 17 may be provided to collect drained fluids.

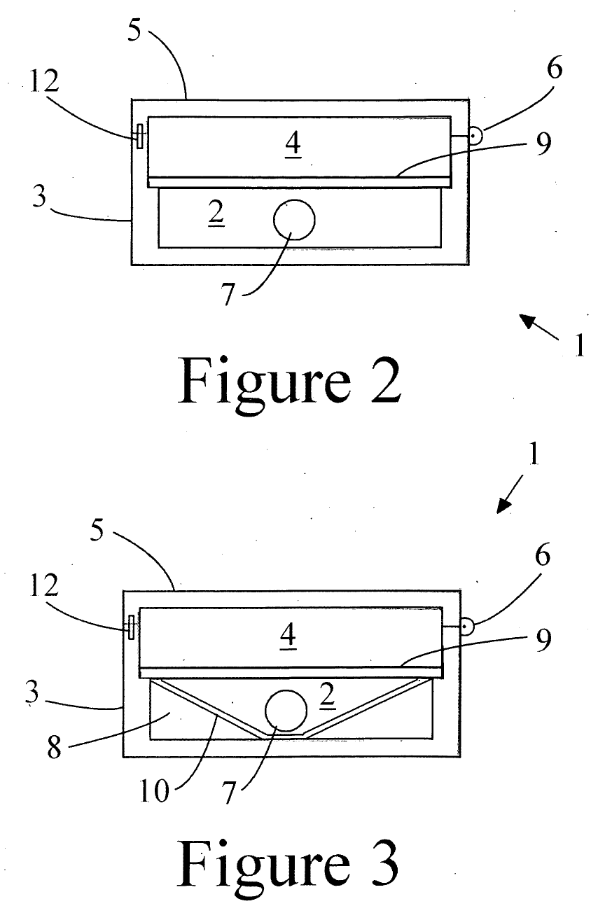

Referring to FIG. 2, there is show a cross-sectional side view of a first embodiment of the sponge sterilizer 1 of the invention. Here, the lid 5 is connected to the housing 3 by hinges 6, but the lid may also be designed to simply be lifted off entirely, thereby further reducing costs of manufacture. It is preferable that an interlock 12 be provided so as to shut off the UV lamp 7 when the lid 5 is opened by the user, so as to prevent exposure of the user to short wave UV radiation. The radiation emitted by the UV lamp may be of wavelengths anywhere from about 200 to about 300 nanometers, generally about 250 nanometers. It is undesirable to expose the human eyes and skin to such wavelengths.

UV lamps are generally expensive, because they must be made of a UV-transparent material, usually quartz. It is desirable, therefore to provide the interior of the sponge chamber 4 with at least one UV-reflective surface, so that valuable UV radiation is not wasted by being absorbed into the container walls. More preferably, the lamp chamber 2 will also have at least one reflective surface for the same reasons. Still more preferable, all of the interior surfaces will be reflective to UV radiation with the exception, of course, of the UV-transmissive support 9. By reflecting the UV radiating within the box, the number of UV lamps 7 required is reduced, the UV lamps 7 being the most expensive single component in the system. Further, the UV radiation is essentially trapped in a “light box,” thereby surrounding and “bathing” the sponge in UV radiation and improving the effectiveness of the radiation's sterilizing effect.

One simple way of providing reflective interior surfaces is to simply manufacture the casing 3 and lid 5 of a reflective material such as a metal. For household use, with such use's repeated exposure to water, stainless steel may be a suitable choice.

An alternative to stainless steel would be to manufacture the casing 3 and lid 5 of a polymer or of glass. The interior surfaces may then be provided with reflective surfaces, such as by inserting metallic elements, such as plates or foils, or by coating the interiors with a UV reflective material, such as by vapor deposition suitable materials for reflecting short wave UV are known in the art of vapor deposition, such as aluminum and its alloys, such as aluminum magnesium fluoride (AlMgF₂), silver-anodized aluminum, and other coatings known in the UV reflective art. Such coatings may be vapor deposited. Wood and ceramics are also suitable materials for the casing 3 and lid 5.

Referring to FIG. 3, there is shown another embodiment of the invention wherein a reflector element 10 is installed next to the UV-lamp 7 in a manner effective in reflecting UV light emitted by the lamp toward the sponge chamber 4. The reflector element 10 also serves the purpose of creating component chambers 8, wherein the electrical components (not shown) needed to activate the UV-lamp may be disposed. In this manner, the electrical components are protected from any water that might drip from the sponge. Further, the reflector may be shaped and inclined to channel any dripping water to the lower drain hole 11b of FIG. 1. It should be noted that the power supply to the UV lamp 7 may be derived from batteries, house voltage, or a combination of both, such as in a battery backup. Small germicidal UV lamps are known that can generally be powered by as few as four AA batteries.

Note also, that by providing a reflector component 10, the interior surfaces of the lamp chamber 2 need not be reflective, as it is the reflector component that serves this function. This can save costs associated with using vapor deposition techniques or expensive materials in the manufacture of the casing 3.

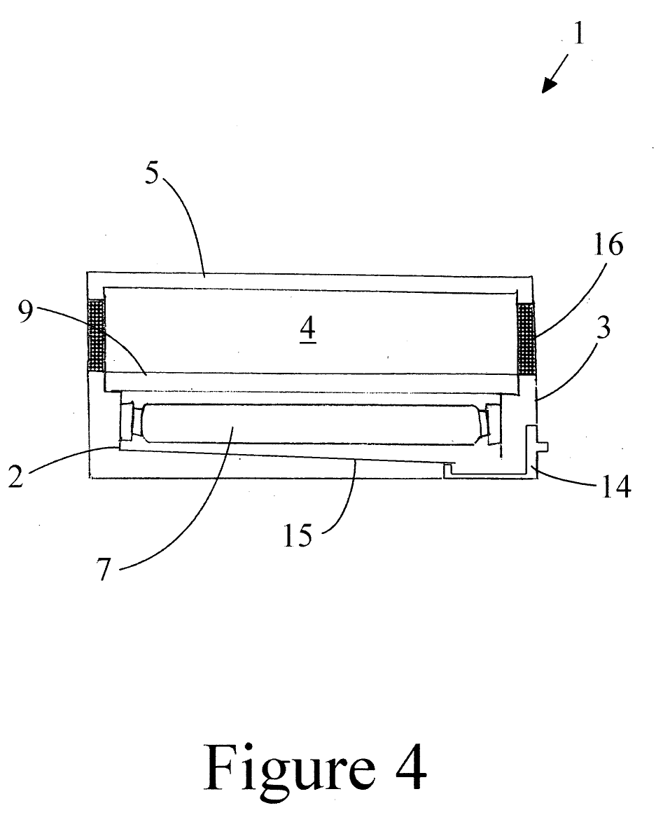

Referring to FIG. 4, there is shown options that may be incorporated with any of the previous embodiments. A sloped low surface 15 in the casing 3 may be provided to direct water to a removable drain tray 14. Additionally, a circumferential glass window 16 may be provided that goes completely or partially around the container. Because it is made of glass, harmful short wave UV radiation is blocked while providing an attractive blue glow that permits the user to see that the device is operational. The circumferential glass window 16 may be vapor deposited with a UV reflective material to improve germicidal effect, yet still transmit light in the visible spectrum.

As can be seen, a relatively effective sponge sterilizer 1 may be provided at low cost by minimizing the use of short wave UV lamps 7 and providing cheaper alternatives to the use of expensive materials.

While various values, scalar and otherwise, may be disclosed herein, it is to be understood that these are not exact values, but rather to be interpreted as “about” such values, unless explicitly stated otherwise. Further, the use of a modifier such as “about” or “approximately” in this specification with respect to any value is not to imply that the absence of such a modifier with respect to another value indicated the latter to be exact.

Changes and modifications can be made by those skilled in the art to the embodiments as disclosed herein and such examples, illustrations, and theories are for explanatory purposes and are not intended to limit the scope of the claims. Further, the abstract of this disclosure is provided for the sole purpose of complying with the rules requiring an abstract so as to allow a searcher or other reader to quickly ascertain the subject matter of the disclosures contained herein and is submitted with the express understanding that it will not be used to interpret or to limit the scope or the meaning of the claims.

1. An apparatus for sterilizing sponges, comprising:
a container, comprising a casing and lid, having a UV-transmissive support structure disposed therein, the UV-transmissive Support dividing the interior of the container into a sponge chamber and a lamp chamber, the sponge chamber dimensioned to receive a sponge;
a source of ultraviolet light disposed within the lamp chamber;
wherein at least one interior surface of the sponge chamber is reflective to UV light; and
wherein the sponge chamber presents no external access while the source of ultraviolet light is active.

2. The apparatus of claim 1 wherein the UV-transmissive support structure comprises quartz.

3. The apparatus of claim 1 wherein the UV-transmissive support structure comprises a grid.

4. The apparatus of claim 1 wherein the casing is made of metal reflective to short wave ultraviolet light.

5. The apparatus of claim 1 wherein the casing interior surfaces are coated with a UV-reflective material.

6. The apparatus of claim 5 wherein the casing comprises glass.

7. The apparatus of claim 5 wherein the casing comprises polymer.

8. The apparatus of claim 1 wherein the lid is made of metal reflective to short wave ultraviolet light.

9. The apparatus of claim 1 wherein the lid interior surfaces are coated with a UV-reflective material.

10. The apparatus of claim 9 wherein the lid comprises glass.

11. The apparatus of claim 9 wherein the lid comprises a polymer.

12. The apparatus of claim 1 wherein the UV lamp emits ultraviolet light at a wavelength of between 200 and 300 nanometers.

13. The apparatus of claim 1 further comprising a reflector element disposed within the lamp chamber so as to reflect light from the UV lamp into the sponge chamber.

14. The apparatus of claim 13 wherein the reflector element is shaped to create one or more component chambers effective in protecting electrical components disposed therein from any liquids dripping from the sponge.

15. The apparatus of claim 1 further comprising an interlock.

16. The apparatus of claim 1 further comprising a circumferential glass window.

17. The apparatus of claim 1 further comprising a removable drain tray.

18. An apparatus for sterilizing sponges, comprising:
a container, comprising a casing and lid, having a UV-transmissive support structure disposed therein, the UV-transmissive support dividing the interior of the container into a sponge chamber and a lamp chamber, the sponge chamber dimensioned to receive a sponge;
a source of ultraviolet light disposed within the lamp chamber;
wherein at least one interior surface of the sponge chamber is reflective to UV light;
further comprising at least one glass window; and
wherein the sponge chamber presents no external access while the source of ultraviolet light is active.
